(19) United States
(12) Patent Application Publication
(10) Pub. No.: US 2023/0393381 A1
(43) Pub. Date: Dec. 7, 2023

(54) LIGHT SOURCE DEVICE, DETECTION UNIT, OPTICAL SYSTEM, ENDOSCOPE, AND INDUSTRIAL MICROSCOPE

(71) Applicant: PANASONIC INTELLECTUAL PROPERTY MANAGEMENT CO., LTD., Osaka (JP)

(72) Inventors: Shintaro HAYASHI, Hyogo (JP); Takeshi ABE, Osaka (JP); Shogo MOTEGI, Osaka (JP); Toshiaki TAKENAKA, Osaka (JP); Fumiya YAGI, Osaka (JP); Natsuki KITO, Kyoto (JP)

(73) Assignee: PANASONIC INTELLECTUAL PROPERTY MANAGEMENT CO., LTD., Osaka (JP)

(21) Appl. No.: 18/033,701

(22) PCT Filed: Oct. 13, 2021
(86) PCT No.: PCT/JP2021/037873

§ 371 Date: Apr. 25, 2023

(30) Foreign Application Priority Data
Oct. 28, 2020 (JP) 2020-180863

(51) Int. Cl. G02B 23/24; G02B 21/06

(52) U.S. Cl. CPC G02B 23/2469; G02B 21/06; A61B 1/00126

(57) ABSTRACT

A light source device includes a connector, a light source, a lever member, and a detector. The connector allows the light guide member to be detachably connected thereto. The light source emits light to be incident on the light guide member connected to the connector. The lever member is displaced depending on presence or absence of the connection of the light guide member to the connector. The detector detects the displacement of the lever member and outputs a detection result.

### TECHNICAL FIELD

The present invention relates to a light source device, a detection unit, an optical system, an endoscope, and an industrial microscope.

### BACKGROUND ART

An endoscope device in PTL 1 includes an endoscope and a controller, and the endoscope and the controller are detachably connected via a connector system.

A leading end of the endoscope is provided with an image capturing portion that captures an optical image and a lighting portion.

The image capturing portion is electrically connected to an image processing device provided in the controller via an electric cable inserted through the endoscope. The image capturing portion operates with power and a drive signal each input thereto from the controller via the electric cable to output a video signal to the controller via the electric cable.

The lighting portion is connected to a light source device provided in the controller via an optical fiber cable inserted through the endoscope to emit light emitted from the light source device toward a subject in the image capturing portion.

Specifically, the light source device includes a receptacle portion and a light source portion. The receptacle portion has a recessed shape that allows a plug portion provided in the endoscope to be inserted in the inside thereof. The receptacle portion is provided on a front surface of the light source device. The light emitted from the light source portion is made incident on the receptacle portion. Then, the plug portion of the endoscope is connected to the receptacle portion to electrically and mechanically connect the controller and the endoscope, whereby the light emitted from the light source portion is transmitted to the endoscope.

In the endoscope device described above in PTL 1, even when the plug portion (light guide member) of the endoscope is not connected to the receptacle portion (connector), the light source portion (light source) is undesirably turned ON. As a result, the light emitted from the light source portion leaks through the receptacle portion to the outside of the light source device.

### CITATION LIST

### Patent Literature

Patent Literature 1 Japanese Patent No. 5149463

### SUMMARY OF INVENTION

An object of the present disclosure is to provide a light source device, a detection unit, an optical system, an endoscope, and an industrial microscope each capable of inhibiting light emitted from a light source from leaking to the outside when a light guide member is not connected to a connector.

A light source device according to an aspect of the present disclosure includes: a connector; a light source; a lever member; and a detector. The connector allows a light guide member to be detachably connected thereto. The light source emits light to be incident on the light guide member connected to the connector. The lever member is displaced depending on presence or absence of the connection of the light guide member to the connector. The detector detects the displacement of the lever member and outputs a detection result.

A detection unit according to an aspect of the present disclosure includes: the lever member and the detector each included in the light source device described above.

An optical system according to an aspect of the present disclosure includes: the light source device described above; and the light guide member.

An endoscope according to an aspect of the present disclosure includes: the light source device described above; and the light guide member.

An industrial microscope according to an aspect of the present disclosure includes: the light source device described above; and the light guide member.

### BRIEF DESCRIPTION OF DRAWINGS

FIG. 1 is a perspective view illustrating an optical system including a light source device according to an embodiment.

FIG. 2 is a perspective view illustrating the same light source device as above.

FIG. 3 is a front view of the same light source device as above.

FIG. 4 is a cross-sectional view along A-A in FIG. 3, which illustrates the same light source device as above.

FIG. 5 is an exploded perspective view illustrating the same light source device as above.

FIG. 6 is a perspective view illustrating a base of the same light source device as above.

FIG. 7 is a perspective view illustrating a light source unit of the same light source device as above.

FIG. 8 is a perspective view illustrating a plate of the same light source device as above.

FIG. 9 is a perspective view of a cover unit of the same light source device as above.

FIG. 10 is a perspective view illustrating a light diffuser, a wavelength converter, and a receptacle of the same light source device as above.

FIG. 11 is a perspective view illustrating a detection unit of the same light source device as above.

FIG. 12 is another perspective view illustrating the same detection unit as above.

FIG. 13 is a side view illustrating a plug unconnected state in the same light source device as above.

FIG. 14 is a side view illustrating a plug connected state in the same light source device as above.

### DESCRIPTION OF EMBODIMENTS

The present embodiment generally relates to a light source device, a detection unit, an optical system, an endoscope, and an industrial microscope. More particularly, the present disclosure relates to a light source device, a detection unit, an optical system, an endoscope, and an industrial microscope to each of which a light guide member is to be detachably connected.

Referring to the drawings, a description will be given below of the light source device, the detection unit, the optical system, the endoscope, and the industrial microscope each according to the embodiment. Each of figures described in the following embodiment and the like is a schematic diagram, and respective ratios of sizes and thicknesses of individual components in the figures do not necessarily reflect actual dimensional ratios.

Note that the embodiment described below is only an example of embodiments of the present disclosure. The present disclosure is not limited to the following embodiment, and various changes may be made depending on design and the like as long as effects of the present disclosure can be achieved.

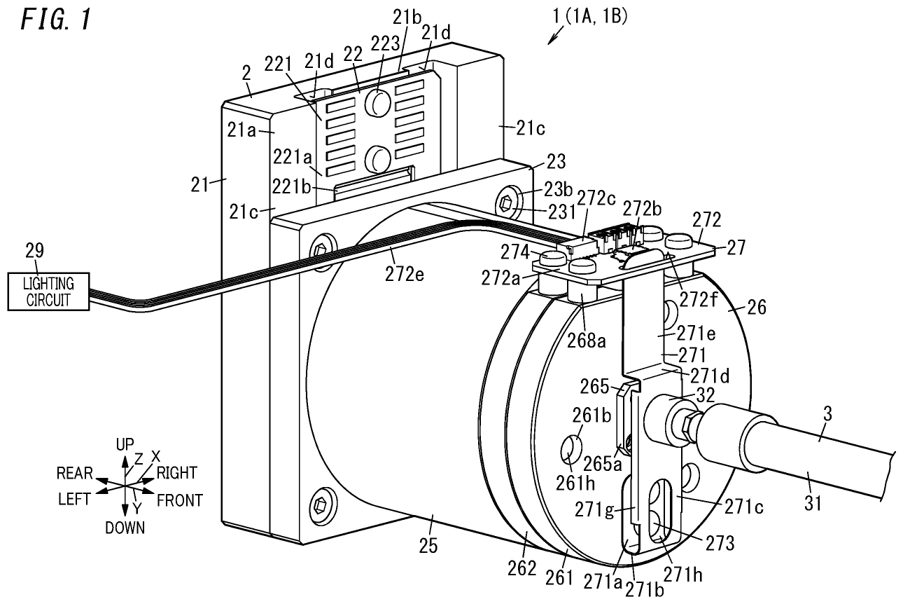

Additionally, in the following description, unless otherwise specified, an X-axis a Y-axis, and a Z-axis that are perpendicular to each other in FIG. 1 are defined. For the sake of convenience, one of two directions along the X-axis is assumed to be a right direction, while another thereof is assumed to be a left direction. One of two directions along the Y-axis is assumed to be a forward direction, while another thereof is assumed to be a rearward direction. One of two directions along the Z-axis is assumed to be an upward direction, while another thereof is assumed to be a downward direction.

### Embodiment

(1) Optical System

An optical system 1 illustrated in FIG. 1 includes a light source device 2 and a light guide member 3. The light source device 2 is connected to the light guide member 3 to output light to the light guide member 3. The light guide member 3 transmits the light output from the light source device 2. In the present embodiment, the light source device 2 outputs laser light (coherent light), and the light guide member 3 transmits the laser light. Examples of the optical system 1 include an endoscope TA for observing the inside of a human body and an industrial microscope 1B for observing metal, a cell, or the like.

(2) Light Source Device

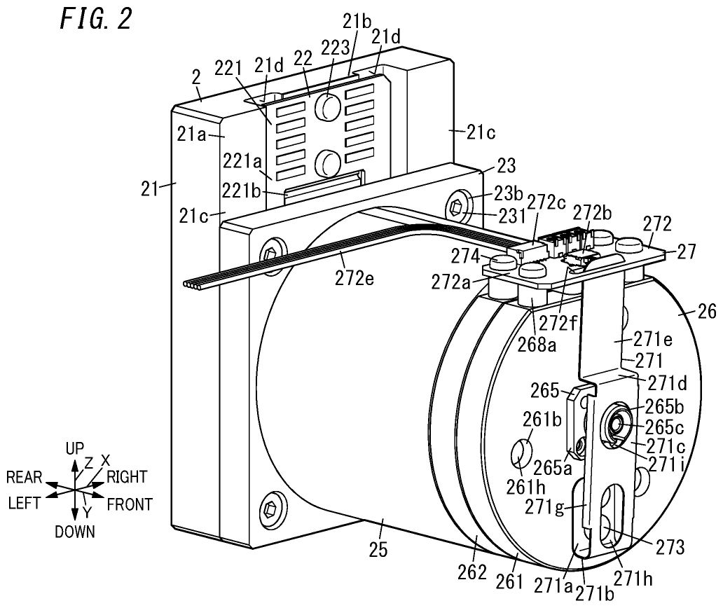

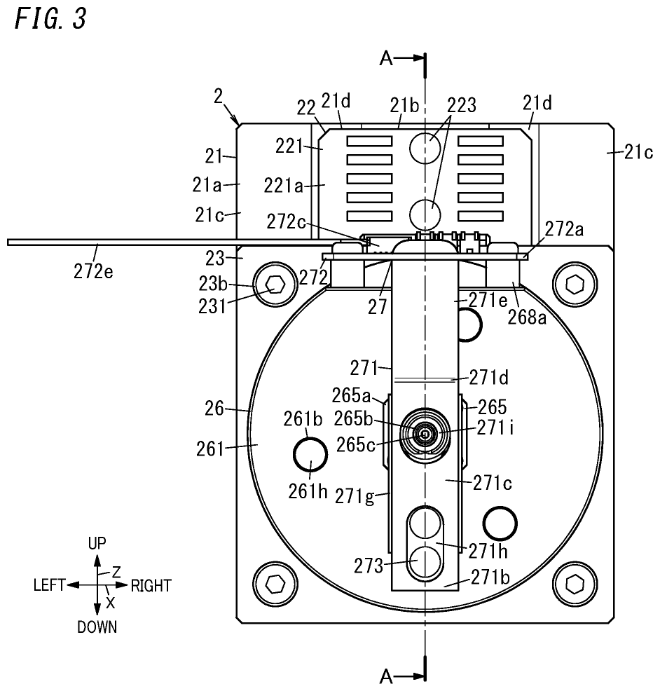

As illustrated in FIGS. 1 to 5, the light source device 2 includes a base 21, a light source unit 22, a plate 23, a lens unit 24, a holder 25, a cover unit 26, a detection unit 27, and a lighting circuit 29.

(2.1) Base

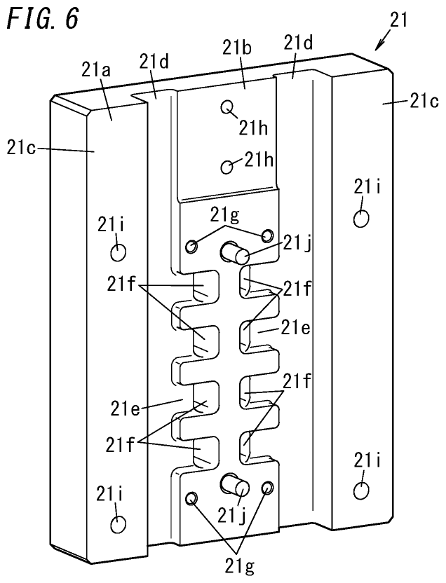

FIG. 6 is a perspective view of the base 21. The base 21 is formed of a metal or resin into a rectangular plate shape. In a front surface 21a of the base 21, a rectangular parallelpiped groove portion 21b extending along an up-down direction along the Z-axis is formed in a middle in a left-right direction along the X-axis. The groove portion 21b is interposed in the left-right direction between a pair of rectangular parallelpiped protruding portions 21c extending along the up-down direction. In each of a left edge and a right edge of the groove portion 21b, a comb-like recessed portion 21d having four comb-tooth portions 21e arranged in the up-down direction is formed, and leading ends of the comb-tooth portions 21e of the left recessed portion 21d and leading ends of the comb-tooth portions 21e of the right recessed portion 21d face each other. Each of the comb-tooth portions 21e has a through hole 21f formed to extend therethrough in a front-rear direction along the Y-axis.

In the groove portion 21b, above and below the respective comb-tooth portions 21e, screw holes 21g are formed. In addition, at a middle of an upper part of the groove portion 21b in the left-right direction, two screw holes 21h arranged in the up-down direction are formed. Meanwhile, in each of the protruding portions 21c, two screw holes 21i arranged in the up-down direction are formed. In other words, in the base 21, the four screw holes 21g, the two screw holes 21h, and the four screw holes 21i are formed.

In addition, at a middle of the groove portion 21b in the left-right direction, two rod-shaped bosses 21j arranged in the up-down direction are provided.

(2.2) Light Source Unit

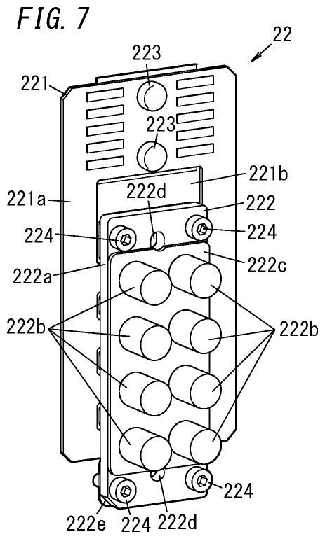

FIG. 7 is a perspective view of the light source unit 22. The light source unit 22 includes a power source circuit 221 and a light source 222.

The power source circuit 221 has a function of feeding a dc power to the light source 222. Specifically, the power source circuit 221 includes a rectangular plate-shaped substrate 221a. The substrate 221a includes an opening 221b. Of the opening 221b, a portion facing the light source 222 is formed in a comb shape. The substrate 221a further includes a conductor (circuit pattern) formed of copper, aluminum, or the like. Further, on the substrate 221a, a circuit element such as a resistor, a capacitor, or a transistor may also be mounted.

The light source 222 includes a rectangular plate-shaped substrate 222a, eight laser diodes (Laser Diodes) 222b (hereinafter abbreviated as LDs 222b), a rectangular plate-shaped packing 222c, and a comb-like plate-shaped packing 222e. The packing 222c is disposed so as to be superimposed on a front surface of the substrate 222a and, on a front surface of the packing 222c, the eight LDs 222b are mounted. On the substrate 222a, two pairs of the four LDs 222b arranged in the up-down direction are mounted to be arranged in the left-right direction. A pair of lead terminals of each of the LDs 222b are inserted through the packing 222c and the substrate 222a to be connected to the conductor of the substrate 222a. The packing 222e is disposed so as to be superimposed on a rear surface of the substrate 222a. When fed with the dc power, each of the LDs 222b emits the laser light.

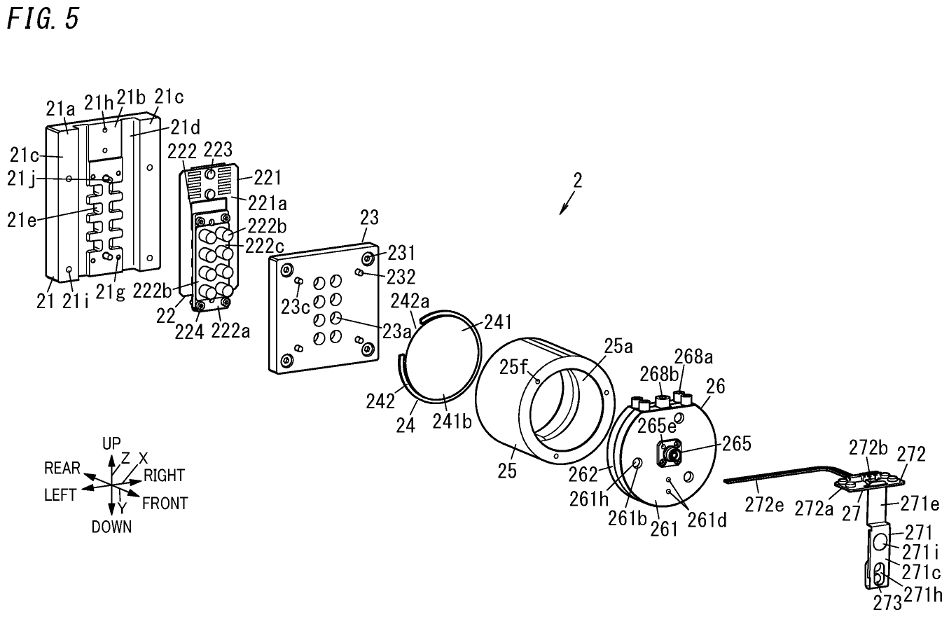

The light source unit 22 is attached to the front surface 21a of the base 21. Specifically, two screws 223 are inserted through two through holes (not shown) of the substrate 221a to be respectively screwed into the two screw holes 21h (see FIGS. 5 and 6) of the base 21. In other words, the power source circuit 221 is fixed to the base 21 by the two screws 223. In addition, four screws 224 are inserted through respective through holes (not shown) formed in four corners of the substrate 222a and respective through holes (not shown) formed in four corners of the packing 222e to be respectively screwed into the four screw holes 21g (see FIGS. 5 and 6) of the base 21. In other words, the light source 222 is fixed to the base 21 by the four screws 224. At this time, the two bosses 21j (see FIGS. 5 and 6) of the base 21 are respectively inserted through two through holes 222d formed in the substrate 222a.

The opening 221b of the substrate 221a is formed in the comb shape so as to be inter-engaged with the comb-like recessed portions 21d (see FIGS. 5 and 6) of the base 21. Consequently, the eight LDs 222b respectively face the eight through holes 21f (see FIGS. 5 and 6) of the base 21.

(2.3) Plate

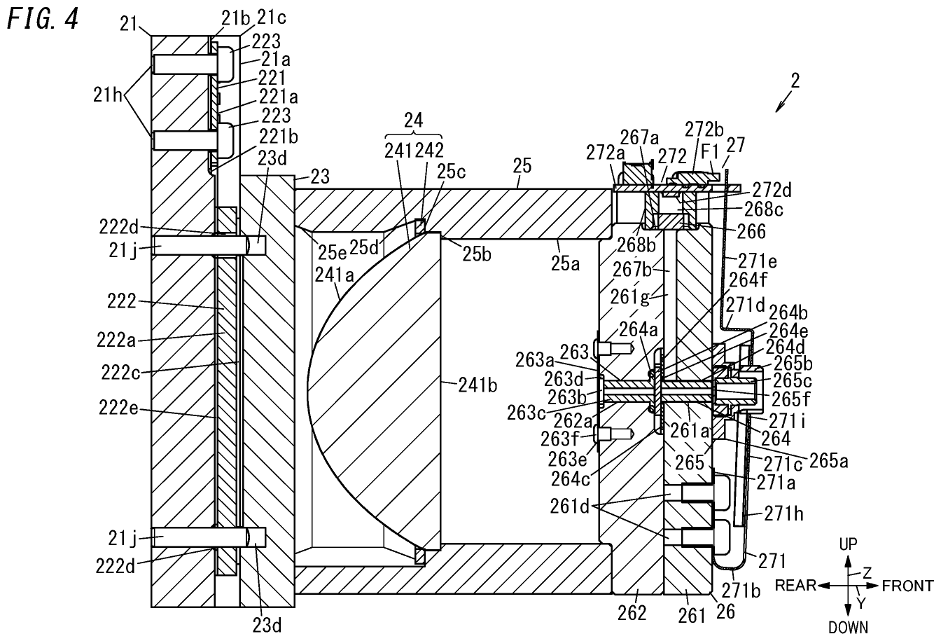

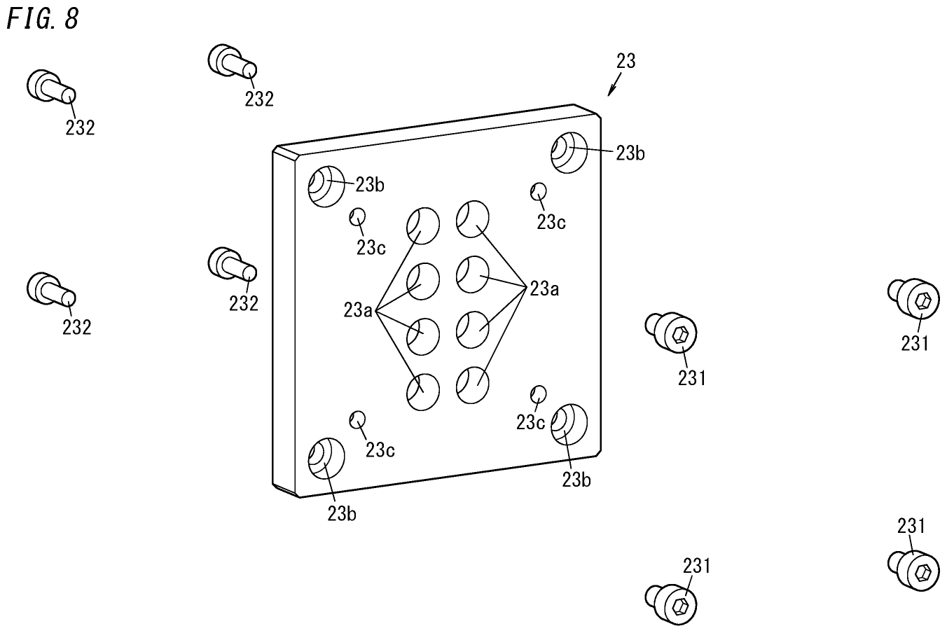

FIG. 8 is a perspective view of the plate 23. The plate 23 is formed of a metal or resin into a rectangular plate shape. In the plate 23, eight through holes 23a, four through holes 23b, and four through holes 23c are formed to extend therethrough in the front-rear direction. In the plate 23, two pairs of the four through holes 23a arranged in the up-down direction are disposed to be arranged in the left-right direction. Four screws 231 are inserted through the respective through holes 23b formed in four corners of the plate 23 to be respectively screwed into the four screw holes 21i (see FIGS. 5 and 6) of the base 21. In other words, the plate 23 is fixed to the base 21 by the four screws 231. At this time, the two bosses 21j (see FIGS. 5 and 6) of the base 21 are respectively inserted through the two through holes 222d of the substrate 222a, and respective leading ends of the two bosses 21j fit in two recessed portions 23d (see FIG. 4) formed in a rear surface of the plate 23. In other words, the plate 23 is positioned by the two bosses 21j. When the plate 23 is fixed to the base 21, the eight LDs 222b of the light source unit 22 are contained in the eight through holes 23a of the plate 23.

(2.4) Lens Unit

As illustrated in FIGS. 4 and 5, the lens unit 24 includes a lens 241 and a ring 242.

The lens 241 is a semispherical capacitor lens. A front surface of the lens 241 is a circular planar emission surface 241b, while a semispherical incidence surface 241a is formed rearward from the emission surface 241b.

The ring 242 is formed of a resin or the like into an arc shape having a notch 242a provided in a portion of a circular ring. The ring 242 is put on a side surface of a front portion of the lens 241 to function as a retainer that prevents the lens 241 from easily coming off the holder 25 described later when the lens 241 is attached to the holder 25.

(2.5) Holder

As illustrated in FIGS. 4 and 5, the holder 25 is formed of a metal or resin into a cylindrical shape. The holder 25 has a cylindrical inner surface 25a, and the inner surface 25a includes a stepped portion 25b, a stepped portion 25c, a tapered surface 25d, and a tapered surface 25e each formed circumferentially around the entire circumference (see FIG. 5).

The stepped portion 25b has a stepped shape in which a front-side diameter of the inner surface 25a is smaller than a rear-side diameter thereof. The stepped portion 25c is located on a rear side of the stepped portion 25b and has a stepped shape in which the front-side diameter of the inner surface 25a is smaller than the rear-side diameter thereof. The tapered surface 25d is located on the rear side of the stepped portion 25c and has a tapered shape in which the diameter of the inner surface 25a gradually increases as it goes forward. The tapered surface 25e is formed on a rear end of the inner surface 25a and has a tapered shape in which the diameter of the inner surface 25a gradually decreases as it goes forward.

When the lens 241 is inserted from the rear side into the holder 25, the emission surface 241b of the lens 241 comes into contact with the stepped portion 25b to restrict forward movement of the lens 241. Then, when the ring 242 is inserted from the rear side into the holder 25, the ring 242 is warped by the tapered surface 25e in a direction in which a diameter of the ring 242 gradually decreases. When the ring 242 moves further forward while staying in contact with the inner surface 25a, the diameter of the ring 242 gradually increases along the tapered surface 25d under an elastic force of the ring 242, and the ring 242 fits into the stepped portion 25c. At this time, the ring 242 is fixed to the stepped portion 25c by being pressed against the inner surface 25a by the elastic force of the ring 242. At this time, the ring 242 is in contact with an outer peripheral edge of the incidence surface 241a of the lens 241 to restrict rearward movement of the lens 241. In other words, the ring 242 functions as a retainer that prevents the lens 241 from easily coming off the holder 25.

The holder 25 is attached to a front surface of the plate 23 by screws 232 (see FIG. 5) respectively inserted from the rear side through the four through holes 23c of the plate 23 by being screwed into screw holes (not shown) formed in a rear end of the holder 25.

(2.6) Cover Unit

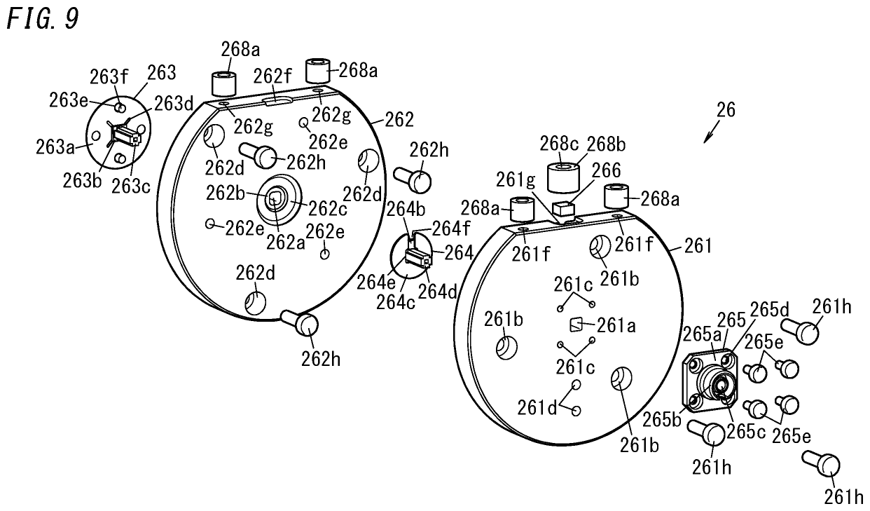

As illustrated in FIG. 9, the cover unit 26 includes a front cover 261, a rear cover 262, a light diffuser 263, a wavelength converter 264, a receptacle 265, and a short pass filter 266.

(2.6.1) Front Cover

The front cover 261 is formed from a metal or resin into a disc shape with a notched top. In the front cover 261, one through hole 261a, three through holes 261b, four screw holes 261c, and two screw holes 261d are formed to extend therethrough in the front-rear direction. The one through hole 261a has a rectangular cross-sectional shape, and is located substantially at a center of the front cover 261. The three through holes 261b are located at 120-degree intervals around the through hole 261a. The four screw holes 261c are located at 90-degree intervals around the through hole 261a. The two screw holes 261d are located below the through hole 261a to be arranged in the up-down direction.

In a rectangular upper surface of the front cover 261, a notched portion 261e and two screw holes 261f are formed. The notched portion 261e is a rectangular notch made forward from a rear end edge of the front cover 261 at a middle of the upper surface of the front cover 261 in the left-right direction. In other words, the notched portion 261e is the notch having an open rear wall. In addition, in a rear surface of the front cover 261, a groove portion 261g having a rectangular cross-sectional shape with open upper and rear surfaces (see FIG. 4) is formed downward from a rear end of the notched portion 261e. The two screw holes 261f are located to be arranged in the left-right direction with the notched portion 261e being interposed therebetween.

(2.6.2) Rear Cover

The rear cover 262 is formed from a metal or resin into a disc shape having a notched top. In the rear cover 262, one through hole 262a, a recessed portion 262b, a recessed portion 262c, three through holes 262d, three screw holes 262e, a notched portion 262f, and two screw holes 262g are formed to extend therethrough in the front-rear direction. The recessed portion 262c is formed substantially at a center of a front surface of the rear cover 262 into a circular cross-sectional shape. The recessed portion 262b is formed in a bottom surface of the recessed portion 262c into a circular cross-sectional shape coaxial with the recessed portion 262c. The one through hole 262a has a rectangular cross-sectional shape, and is formed at a center of a bottom surface of the recessed portion 262b. The three through holes 262d are located at 120-degree intervals around the through hole 262a. The three screw holes 262e are located at 120-degree intervals around the through hole 262a.

In a rectangular upper surface of the rear cover 262, the notched portion 262f and the two screw holes 262g are formed. The notched portion 262f is a rectangular notch made rearward from a front end edge of the rear cover 262 at a middle of the upper surface of the rear cover 262 in the left-right direction. In other words, the notched portion 262f is a notch with an open front wall. The two screw holes 262g are located to be arranged in the left-right direction with the notched portion 262f being interposed therebetween.

The rear cover 262 is attached to a front end of the holder 25 by three screws 262h inserted from a front side through the three through holes 262d by being screwed into screw holes 25f formed in the front end of the holder 25.

Meanwhile, the front cover 261 is attached to the rear cover 262 such that the rear surface of the front cover 261 faces a front surface of the rear cover 262. The front cover 261 is attached to the front surface of the rear cover 262 by three screws 261h inserted from the front side through the three through holes 261b by being screwed into the screw holes 262e formed in the front surface of the rear cover 262.

(2.6.3) Light Diffuser

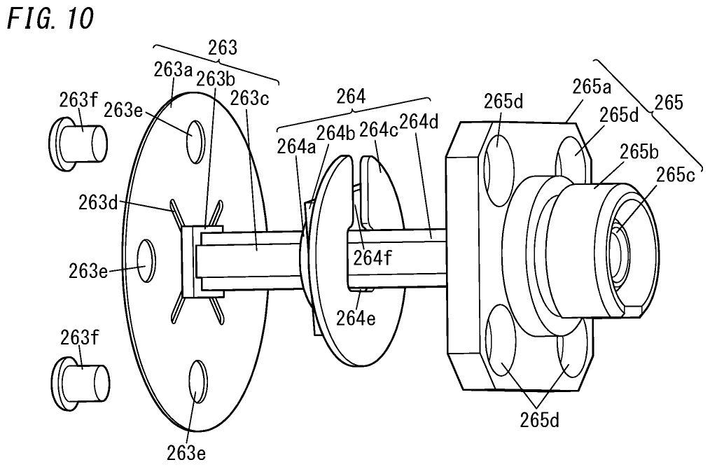

As illustrated in FIG. 10, the light diffuser 263 includes a plate spring 263a, a diffusion plate 263b, and a light tunnel 263c.

The plate spring 263a has a disc shape and, in a center of the plate spring 263a, an X-shaped opening 263d is formed. The diffusion plate 263b has a rectangular plate shape, and is disposed at a center of a front surface of the plate spring 263a. In other words, a rear surface of the diffusion plate 263b is in contact with a front surface of the plate spring 263a, and faces a center of the X-shaped opening 263d. The light tunnel 263c is formed in an elongated rectangular parallelpiped shape, and has a hollow light guide path along a longitudinal direction. The light guide path has a rectangular cross-sectional shape and, over an inner wall of the light guide path, a reflection film is formed. A rear end of the light tunnel 263c is in contact with a center of a front surface of the diffusion plate 263b.

The plate spring 263a is attached to a center of a rear surface of the rear cover 262 by rivets 263f respectively inserted through two through holes 263e, which are among the four through holes 263e and located at intervals of at least 180 degrees. At this time, the light tunnel 263c is inserted from the rear side through the through hole 262a of the rear cover 262, and a front end of the light tunnel 263c is located on the front side of the through hole 262a.

(2.6.4) Wavelength Converter

As illustrated in FIG. 10, the wavelength converter 264 includes an O-ring 264a, a phosphor plate 264b, a C-ring 264c, and a light tunnel 264d. The phosphor plate 264b is a plate having a rectangular plate shape and containing a phosphor. At a center of a rear surface of the phosphor plate 264b, the O-ring 264a is disposed. The O-ring 264a is formed of a resin into an annular shape to be used for sealing. At a center of a front surface of the phosphor plate 264b, the C-ring 264c is disposed. The C-ring 264c is formed in a disc shape and, in a center of the C-ring 264c, a rectangular through hole 264e is formed. In the C-ring 264c, a notch 264f is formed to extend upward from the through hole 264e. The light tunnel 264d is formed in an elongated rectangular parallelpiped shape, and has a hollow light guide path along the longitudinal direction. The light guide path has a rectangular cross-sectional shape and, over an inner wall of the light guide path, a reflection film is formed. The light tunnel 264d has a rear end inserted through the through hole 264e of the C-ring 264c to come into contact with a center of the front surface of the phosphor plate 264b.

The phosphor plate 264b contains the phosphor for converting the laser light emitted from each of the LDs 222b to white laser light. For example, when the laser light emitted from the LD 222b is blue, the phosphor plate 264b contains the phosphor that converts the blue laser light to yellow laser light.

As illustrated in FIG. 9, the wavelength converter 264 is disposed on the front surface of the rear cover 262 such that the C-ring 264c fits into the recessed portion 262c of the rear cover 262. At this time, the O-ring 264a (see FIG. 10) fits into the recessed portion 262b of the rear cover 262. Meanwhile, the light tunnel 264d is inserted from the rear side through the through hole 261a of the front cover 261, and a front end of the light tunnel 264d is located on the front side of the through hole 261a.

(2.6.5) Receptacle (Connector)

The receptacle 265 corresponds to a connector in the present disclosure. As illustrated in FIG. 10, the receptacle 265 is a so-called FC-type receptacle including a flange 265a having a square plate shape, a cylindrical attachment portion 265b, and a cylindrical sleeve 265c. At a center of a front surface of the flange 265a, the attachment portion 265b is provided and, inside the attachment portion 265b, the sleeve 265c is provided coaxially with the attachment portion 265b. At a center of the flange 265a, a through hole 265f (see FIG. 4) is formed to extend in the front-rear direction, and the through hole 265f communicates with the inside of the sleeve 265c.

As illustrated in FIG. 9, four screws 265e are inserted through respective through holes 265d formed in four corners of the sleeve 265c to be respectively screwed into four screw holes 261c formed in a front surface of the front cover 261. In other words, the receptacle 265 is fixed to the front surface of the front cover 261 by the four screws 265e. At this time, a front end of the light tunnel 264d comes into contact with a center of a rear surface of the flange 265a, and the front end of the light tunnel 264d faces the through hole 265f.

(2.6.6) Short Pass Filter

When the front cover 261 is attached to the rear cover 262, the notched portion 261e and the notched portion 262f form a recessed portion 267a (see FIG. 4). In addition, when the front cover 261 is attached to the rear cover 262, a light guide path 267b defined by the groove portion 261g and the front surface of the rear cover 262 is formed (see FIG. 4). A lower portion of the light guide path 267b faces a notch 264f of the C-ring 264c. An opening in an upper end of the light guide path 267b is formed in the bottom surface of the recessed portion 267a.

The short pass filter 266 is disposed on the bottom surface of the recessed portion 267a. At this time, a lower surface of the short pass filter 266 faces the opening in the upper end of the light guide path 267b. The short pass filter 266 has a function of transmitting laser light at a frequency less than a cutoff frequency and attenuating laser light at a frequency equal to or more than the cutoff frequency.

(2.7) Laser Light

The laser light emitted forward from each of the eight LDs 222b is incident on the incidence surface 241a of the lens 241. The lens 241 has a light condensing function of forming an image with the laser light emitted from the LD 222b at a focal point and, from the emission surface 241b of the lens 241, the condensed laser light is emitted forward.

The laser light emitted forward from the emission surface 241b of the lens 241 passes through the holder 25 to be focused on the opening 263d of the plate spring 263a and incident on the rear surface of the diffusion plate 263b. When the laser light is incident on the rear surface of diffusion plate 263b, the diffusion plate 263b emits the laser light as diffused light from the front surface thereof. The laser light diffused by the diffusion plate 263b is incident on a rear end of the light tunnel 263c. The light tunnel 263c emits the laser light having a uniform brightness distribution from a front end of the light tunnel 263c.

The laser light (e.g., the blue laser light) emitted forward from the front end of the light tunnel 263c passes through the phosphor plate 264b to be converted to the white laser light. The laser light having passed through the phosphor plate 264b is incident on the rear end of the light tunnel 264d. The light tunnel 264d emits the laser light having a uniform brightness distribution from the front end of the light tunnel 264d.

The laser light emitted forward from the front end of the light tunnel 264d is incident on a rear end of the sleeve 265c through the through hole 265f of the receptacle 265 to be emitted from a front end of the sleeve 265c.

The laser light diffused by the diffusion plate 263b passes through the notch 264f of the C-ring 264c to be incident on a lower end of the light guide path 267b (see FIG. 4). The laser light incident on the lower end of the light guide path 267b passes through the light guide path 267b to be incident on the lower surface of the short pass filter 266 through the opening in the upper end of the light guide path 267b. The short pass filter 266 emits, from an upper surface of the short pass filter 266, the laser light at the frequency less than the cutoff frequency, which is included in the incident laser light.

(2.8) Detection Unit

As described above, from the receptacle 265 provided on a front surface of the light source device 2, the laser light is emitted forward. Accordingly, to the receptacle 265, the light guide member 3 is connected. The light guide member 3 incudes an optical cable 31 in which one or more optical fibers are contained and a plug 32 attached to a first end of the optical cable 31. The plug 32 is a FC-type plug connectable to the FC-type receptacle 265. In other words, the receptacle 265 and the plug 32 are included in an FC-type optical connector. To a second end of the optical cable 31, an optical member not shown is attached, and the laser light emitted by the light source device 2 from the receptacle 265 propagates through the optical cable 31 and passes through the optical member to be emitted from the second end of the optical cable 31.

However, when the light source 222 is turned ON while the light guide member 3 is not connected to the receptacle 265, the laser light is undesirably emitted from the receptacle 265 to the outside. At this time, unintended laser light illumination may occur.

Accordingly, the light source device 2 includes the detection unit 27.

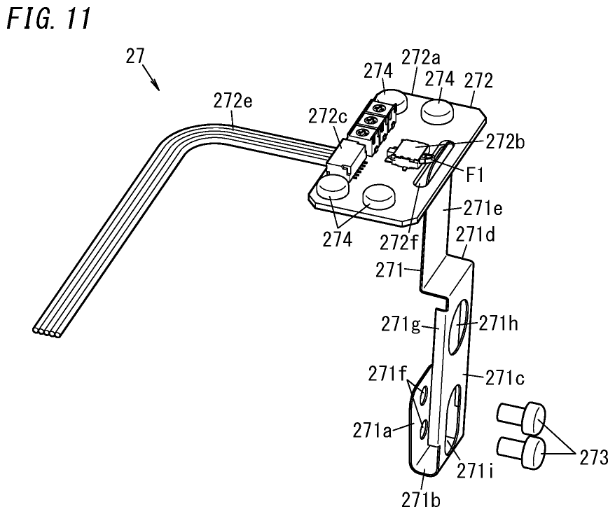

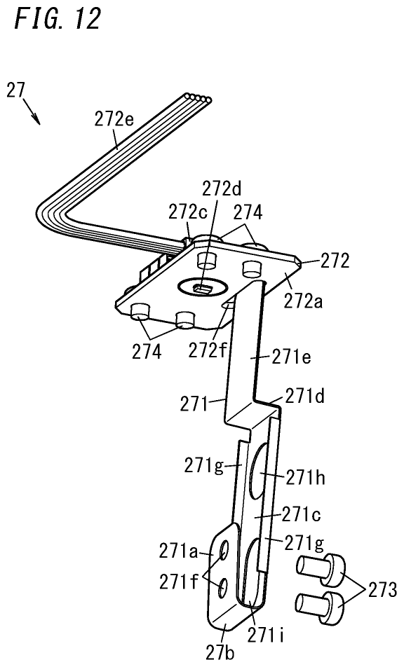

As illustrated in FIGS. 11 and 12, the detection unit 27 includes a lever member 271 and a detector 272.

The lever member 271 is formed by sheet-metal working of a metal such as aluminum, stainless steel, or iron into a J-shape. The lever member 271 includes a fixation piece 271a, a curved portion 271b, an extension piece 271c, a bent portion 271d, and an operation piece 271e.

The fixation piece 271a is a plate-shaped short piece extending in the up-down direction, and has two through holes 271f arranged in the up-down direction. The curved portion 271b is curved forward from a lower end of the fixation piece 271a to be continued to a lower end of the extension piece 271c. The extension piece 271c is a long piece having a rectangular plate shape extending in the up-down direction, and has rearwardly bent ribs 271g respectively formed at left and right side edges thereof. In an upper part of the extension piece 271c, a circular through hole 271h is formed while, in a lower part of the extension piece 271c, an oval through hole 271i elongated in the up-down direction is formed. The two through holes 271f and the through hole 271i face each other in the front-rear direction. The bent portion 271d is bent rearward from an upper end of the extension piece 271c to be continued to a lower end of the operation piece 271e. The operation piece 271e is a long piece having a rectangular plate shape extending in the up-down direction.

The lever member 271 is attached to the front surface of the front cover 261 by screws 273 respectively inserted from the rear side through the through hole 271i and the two through holes 271f by being screwed into the two screw holes 261d (see FIG. 9) formed in the front surface of the front cover 261. Through the through hole 271h of the lever member 271 attached to the front surface of the front cover 261, the attachment portion 265b of the receptacle 265 is inserted from the rear side.

The detector 272 includes a substrate 272a, a detection switch 272b, a connector 272c, an illuminance sensor 272d, and an electric cable 272e.

The substrate 272a has a rectangular plate shape, and includes a conductor (circuit pattern) formed of copper, aluminum, or the like. On an upper surface of the substrate 272a, elements such as the detection switch 272b and the connector 272c are mounted. On a lower surface of the substrate 272a, the illuminance sensor 272d is mounted.

The detection switch 272b includes a contact maker F1. The contact maker F1 is located at a home position unless a force is applied thereto to protrude forward from a front surface of the detection switch 272b (see FIGS. 11 and 12). The contact maker F1 moves rearward when a rearward force is applied thereto, and returns to the home position under a spring force or the like when the rearward force is no longer present. The detection switch 272b has an internal contact that is turned OFF when the contact maker F1 is located at the home position and is turned ON when the contact maker F1 is moving rearward, and the contact is electrically connected to the conductor of the substrate 272a. In other words, the detection switch 272b can output a contact signal according to a displacement of the contact maker F1.

The connector 272c is disposed along a rear edge of the substrate 272a. To a rear surface of the connector 272c, a first end of the multiconductor electric cable 272e is detachably connected. A second end of the electric cable 272e is connected to the lighting circuit 29 (see FIG. 1).

The substrate 272a includes, on the front side of the detection switch 272b, a rectangular opening 272f elongated in the left-right direction. Above the opening 272f, a front end of the contact maker F1 located at the home position is located. Through the opening 272f, a leading end of the operation piece 271e of the lever member 271 is inserted from below. The leading end of the operation piece 271e is located on the front side of the contact maker F1.

The substrate 272a of the detector 272 is placed over four cylindrical spacers 268a disposed on an upper surface of the cover unit 26 and an upper end of one cylindrical body 268b. Then, four screws 274 are inserted through respective through holes (not shown) formed in four corners of the substrate 272a and respective cylinders of the four spacers 268a to be respectively screwed into the two screw holes 261f and the two screw holes 262g (see FIG. 9) of the cover unit 26. In other words, the detector 272 is fixed by the four screws 274 to the cover unit 26.

At this time, as illustrated in FIG. 4, a lower end of the cylindrical body 268b fits into the recessed portion 267a of the upper surface of the cover unit 26, and the short pass filter 266 is contained inside the cylindrical body 268b. The upper end of the cylindrical body 268b comes into contact with the lower surface of the substrate 272a, and the illuminance sensor 272d is contained inside the cylindrical body 268b. In other words, inside the cylindrical body 268b, the short pass filter 266 and the illuminance sensor 272d face each other in the up-down direction, and the laser light having passed through the light guide path 267b and transmitted by the short pass filter 266 illuminates the illuminance sensor 272d. The illuminance sensor 272d measures the presence or absence of the laser light emitted from the light source 222 as well as an intensity of the laser light, and outputs a measurement result. An output terminal of the illuminance sensor 272d is electrically connected to the conductor of the substrate 272a. The substrate 272a outputs the contact signal from the detection switch 272b and the measurement result from the illuminance sensor 272d to the lighting circuit 29 via the connector 272c and the electric cable 272e.

(2.9) Lighting Circuit

The lighting circuit 29 controls the dc power to be fed to the power source circuit 221 of the light source unit 22. In other words, the lighting circuit 29 performs output control including stopping of the laser light emitted from the light source 222.

Specifically, the lighting circuit 29 receives each of the contact signal from the detection switch 272b and the measurement result from the illuminance sensor 272d via the electric cable 272e. Then, the lighting circuit 29 controls the dc power to be fed to the power source circuit 221 of the light source unit 22 on the basis of each of the contact signal from the detection switch 272b and the measurement result from the illuminance sensor 272d. In other words, the lighting circuit 29 controls the output of the laser light on the basis of each of the contact signal from the detection switch 272b and the measurement result from the illuminance sensor 272d.

Specifically, when determining that the contact of the detection switch 272b is OFF (when determining that the light guide member 3 is not connected to the receptacle 265) on the basis of the contact signal from the detection switch 272b, the lighting circuit 29 sets to 0 the dc power to be fed to the power source circuit 221 of the light source unit 22 to stop the output of the laser light.

Meanwhile, when determining that the contact of the detection switch 272b is ON (when determining that the light guide member 3 is connected to the receptacle 265), the lighting circuit 29 is allowed to feed the dc power to the power source circuit 221 of the light source unit 22. Then, when receiving an instruction to output the laser light, the lighting circuit 29 feeds the dc power to the power source circuit 221 and controls the intensity of the laser light to an objective intensity on the basis of the measurement result from the illuminance sensor 272d.

Note that the lighting circuit 29 is preferably a switching power source circuit having a power factor improving function. For example, the switching power source circuit has an AC/DC conversion circuit and a DC/DC conversion circuit. The AC/DC conversion circuit is preferably a boost chopper circuit or a step-up/step-down chopper circuit having the power factor improving function. The DC/DC conversion circuit is preferably a constant-current controlled chopper circuit.

(3) Operation of Detection Unit

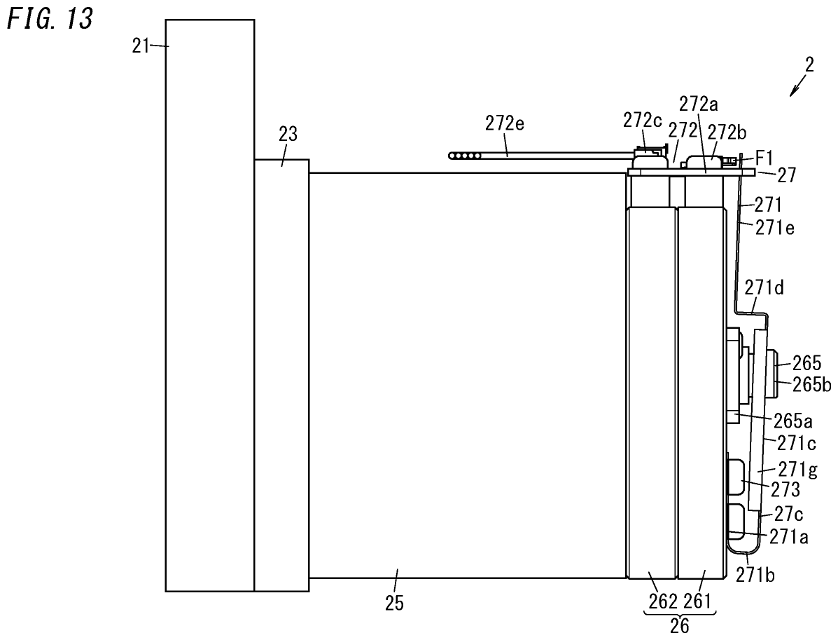

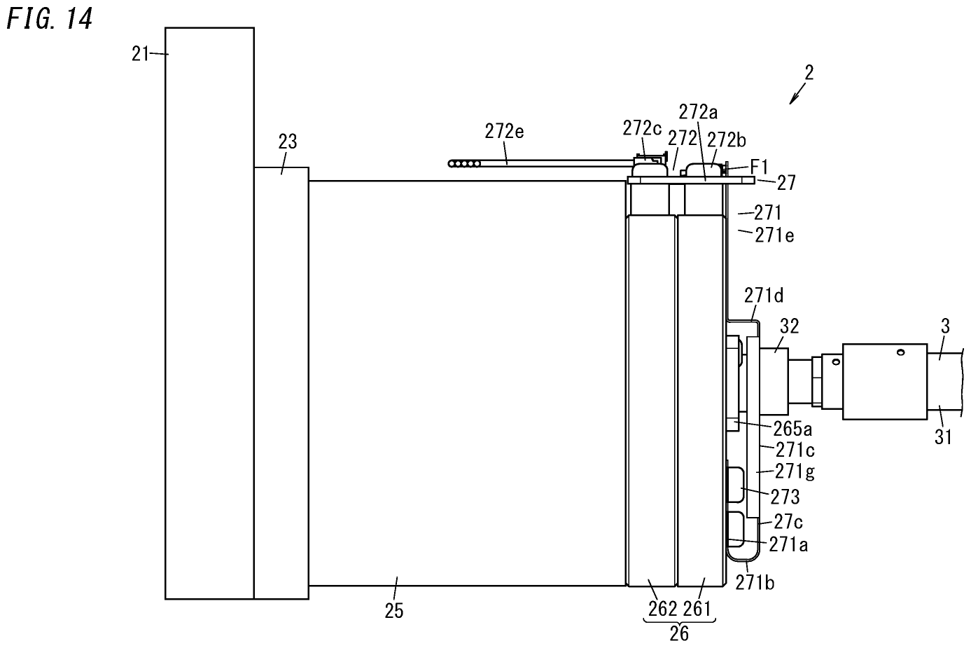

Using FIGS. 13 and 14, a description will be given of an operation of the detection unit 27.

In the lever member 271 of the detection unit 27, the fixation piece 271a is fixed to a front surface of the cover unit 26 by the screws 273, and the curved portion 271b of the lever member 271 functions as a fixed end. The leading end of the operation piece 271e is located on the front side of the contact maker F1 of the detection switch 272b, and the leading end of the operation piece 271e functions as a free end.

FIG. 13 illustrates a state of the detection unit 27 when the plug 32 of the light guide member 3 is not connected to the receptacle 265. When the plug 32 of the light guide member 3 is not connected to the receptacle 265, the extension piece 271c of the lever member 271 is inclined forward, and the operation piece 271e is also inclined forward. Consequently, the leading end of the operation piece 271e is located forwardly away from the contact maker F1 without abutting on the contact maker F1. At this time, the contact maker F1 is located at the home position, the contact of the detection switch 272b is OFF, and the lighting circuit 29 determines that the light guide member 3 is not connected to the receptacle 265. Accordingly, the lighting circuit 29 sets the dc power to be fed to the power source circuit 221 of the light source unit 22 to 0 to stop the output of the laser light. Thus, when the plug 32 of the light guide member 3 is not connected to the receptacle 265, the light source 222 is not turned ON to be able to inhibit the laser light from being emitted from the receptacle 265 to the outside.

FIG. 14 illustrates a state of the detection unit 27 when the plug 32 of the light guide member 3 is connected to the receptacle 265. When the plug 32 of the light guide member 3 is connected to the receptacle 265, a leading end of the plug 32 abuts on a front surface of the extension piece 271c to apply a rearward force to the extension piece 271c. When the rearward force is applied to the extension piece 271c, the extension piece 271c is displaced rearward. When the extension piece 271c is displaced rearward, the operation piece 271e is also displaced rearward, and the operation piece 271e is along the up-down direction. As a result, the leading end of the operation piece 271e abuts on the contact maker F1 from the front side, and the contact maker F1 is moved rearward. At this time, the contact maker F1 is located on the rear side of the home position, the contact of the detection switch 272b is ON, and the lighting circuit 29 determines that the light guide member 3 is connected to the receptacle 265. This allows the lighting circuit 29 to feed the dc power to the power source circuit 221 of the light source unit 22.

When the plug 32 is connected to the receptacle 265, a spring force to displace the extension piece 271c forward by using the curved portion 271b as the fixed end is exerted thereon. As a result, when the plug 32 is detached from the receptacle 265, the extension piece 271c is displaced forward, the operation piece 271e is also displaced forward, and the extension piece 271c and the operation piece 271e are inclined forward to return the contact maker F1 to the home position (see FIG. 13).

In addition, the leading end of the operation piece 271e is inserted through the opening 272f of the substrate 272a. In other words, the displacement of the operation piece 271e in the left-right direction and the front-rear direction is limited by the opening 272f. This can inhibit the swing, displacement, or the like of the operation piece 271e and facilitates positioning of the operation piece 271e.

(4) Modification

The detector 272 may also include, as a sensor that detects a position of the lever member 271 in a non-contact manner, an optical sensor, a magnetic sensor, or the like. In this case, the contact of the detection unit is turned ON or OFF depending on a detection result from the sensor. By detecting the position of the lever member 271 in a non-contact manner, it is possible to suppress mechanical abrasion of the detector 272.

The light source 222 may also include, instead of the LDs 222b, an LED (Light Emitting Diode), an organic EL (Organic Electro Luminescence or OEL), or the like and output non-coherent light (such as visible light or infrared light). In this case, the light output from the light source device 2 is also non-coherent light.

The lighting circuit 29 may be either configured integrally with the power source circuit 221 or configured separately from the power source circuit 221.

The substrate 272a may also include a notch instead of the opening 272f.

The substrate 272a may also include, instead of the opening 272f, a spring member having an insertion portion through which the lever member 271 is inserted. The spring member is configured to include a spring terminal that can be mounted on a printed substrate or the like.

(5) Summary

A light source device (2) in a first aspect according to the embodiment described above includes a connector (265), a light source (222), a lever member (271), and a detector (272). The connector (265) is detachably connected to a light guide member (3). The light source (222) emits light to be incident on the light guide member (3) connected to the connector (265). The lever member (271) is displaced depending on the presence or absence of the connection of the light guide member (3) to the connector (265). The detector (272) detects the displacement of the lever member (271), and outputs a detection result.

The light source device (2) described above can inhibit the light emitted from the light source (222) from leaking to the outside when the light guide member (3) is not connected to the connector (265).

The light source device (2) in a second aspect according to the embodiment described above preferably further includes a lighting circuit (29) in the first aspect. The lighting circuit (29) determines, on the basis of the detection result, whether or not the light guide member (3) is connected to the connector (265), and turns OFF the light source (222) when determining that the light guide member (3) is not connected to the connector (265).

In the light source device (2) described above, the lighting circuit (29) operates on the basis of the detection result from the detector (272) to be able to inhibit the light emitted from the light source (222) from leaking to the outside when the light guide member (3) is not connected to the connector (265).

In the light source device (2) in a third aspect according to the embodiment described above, the detector (272) preferably includes, in the first or second aspect, a contact maker (F1) with which the lever member (271) comes into contact when the light guide member (3) is connected to the connector (265).

The light source device (2) described above can more reliably detect the displacement of the lever member (271).

In the light source device (2) in a fourth aspect according to the embodiment described above, in the first or second aspect, the detector (272) preferably includes a sensor that detects a position of the lever member (271) in a non-contact manner.

The light source device (2) described above can inhibit mechanical abrasion of the detector (272).

In the light source device (2) in a fifth aspect according to the embodiment described above, the detector (272) preferably includes, in any one of the first to fourth aspects, a substrate (272a) having an opening (272f) or a notch through which the lever member (271) is inserted.

The light source device (2) described above can inhibit a swing, displacement, or the like of the lever member (271), and facilitates positioning of the lever member (271).

In the light source device (2) in a sixth aspect according to the embodiment described above, the detector (272) preferably includes, in any one of the first to fourth aspects, a spring member having an insertion portion through which the lever member (271) is inserted.

The light source device (2) described above can inhibit a swing, displacement, or the like of the lever member (271), and facilitates positioning of the lever member (271).

In the light source device (2) in a seventh aspect according to the embodiment described above, it is preferable in any one of the first to sixth aspects that the lever member (271) has one end as a fixed end, has another end as a free end, and is displaced by movement of the free end.

The light source device (2) described above can easily implement the displaceable lever member (271).

In the light source device (2) in an eighth aspect according to the embodiment described above, the light source (222) preferably emits laser light as the light in any one of the first to seventh embodiments.

The light source device (2) described above can inhibit the laser light from leaking to the outside.

A detection unit (27) in a ninth aspect of the embodiment described above includes the lever member (271) and the detector (272) each included in the light source device (2) according to any one of the first to eighth aspects.

The detection unit (27) described above can inhibit the light emitted from the light source (222) from leaking to the outside when the light guide member (3) is not connected to the connector (265).

An optical system (1) in a tenth aspect according to the embodiment described above includes the light source device (2) according to any one of the first to eighth aspects and the light guide member (3).

The optical system (1) described above can inhibit the light emitted from the light source (222) from leaking to the outside when the light guide member (3) is not connected to the connector (265).

An endoscope (TA) in an eleventh aspect according to the embodiment described above includes the light source device (2) according to any one of the first to eighth aspects and the light guide member (3).

The endoscope (TA) described above can inhibit the light emitted from the light source (222) from leaking to the outside when the light guide member (3) is not connected to the connector (265).

An industrial microscope (1B) in a twelfth aspect according to the embodiment described above includes the light source device (2) according to any one of the first to eighth aspects and the light guide member (3).

The industrial microscope (1B) described above can inhibit the light emitted from the light source (222) from leaking to the outside when the light guide member (3) is not connected to the connector (265).

### REFERENCE SIGNS LIST

1 Optical system
1A Endoscope
1B Industrial microscope
2 Light source device
222 Light source
265 Receptacle (Connector)
27 Detection unit
271 Lever member
272 Detector
272a Substrate
272f Opening
29 Lighting circuit
3 Light guide member
F1 Contact maker

1. A light source device comprising:
a connector that allows a light guide member to be detachably connected thereto;
a light source that emits light to be incident on the light guide member connected to the connector;
a lever member that is displaced depending on presence or absence of the connection of the light guide member to the connector; and
a detector that detects the displacement of the lever member and outputs a detection result.

2. The light source device according to claim 1, further comprising:
a lighting circuit that determines, on the basis of the detection result, whether or not the light guide member is connected to the connector and turns OFF the light source when determining that the light guide member is not connected to the connector.

3. The light source device according to claim 1, wherein the detector includes a contact maker, with which the lever member is brought into contact, when the light guide member is connected to the connector.

4. The light source device according to claim 1, wherein the detector includes a sensor that detects a position of the lever member in a non-contact manner.

5. The light source device according to claim 1, wherein the detector includes a substrate having an opening or a notch through which the lever member is inserted.

6. The light source device according to claim 1, wherein the detector includes a spring member having an insertion portion through which the lever member is inserted.

7. The light source device according to claim 1, wherein the lever member has one end as a fixed end, has another end as a free end, and is displaced by movement of the free end.

8. The light source device according to claim 1, wherein the light source emits laser light as the light.

9. A detection unit comprising:
the lever member and the detector each included in the light source device according to claim 1.

10. An optical system comprising:
the light source device according to claim 1; and
the light guide member.

11. An endoscope comprising:
the light source device according to claim 1; and
the light guide member.

12. An industrial microscope comprising:
the light source device according to claim 1; and
the light guide member.

13. The light source device according to claim 2, wherein the detector includes a contact maker, with which the lever member is brought into contact, when the light guide member is connected to the connector.

14. The light source device according to claim 2, wherein the detector includes a sensor that detects a position of the lever member in a non-contact manner.

15. The light source device according to claim 2, wherein the detector includes a substrate having an opening or a notch through which the lever member is inserted.

16. The light source device according to claim 3, wherein the detector includes a substrate having an opening or a notch through which the lever member is inserted.

17. The light source device according to claim 4, wherein the detector includes a substrate having an opening or a notch through which the lever member is inserted.

18. The light source device according to claim 2, wherein the detector includes a spring member having an insertion portion through which the lever member is inserted.

19. The light source device according to claim 3, wherein the detector includes a spring member having an insertion portion through which the lever member is inserted.

20. The light source device according to claim 4, wherein the detector includes a spring member having an insertion portion through which the lever member is inserted.
